(19) United States
(12) Patent Application Publication
(10) Pub. No.: US 2007/0092870 A1
(43) Pub. Date: Apr. 26, 2007

(54) Detection of biomolecules

(76) Inventors: Yiping Zhao, Statham, GA (US); Ralph A. Tripp, Watkinsville, GA (US); Junxue Fu, Athens, GA (US); Les Jones, Watkinsville, GA (US); Rene Alvarez, Athens, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/584,072

(22) Filed: Oct. 20, 2006

Related U.S. Application Data
(60) Provisional application No. 60/728,572, filed on Oct. 20, 2005.

(51) Int. Cl. C12Q 1/70; C12Q 1/68; C07H 21/04

(52) U.S. Cl. 435/5; 435/6; 536/23.72; 977/924

(57) ABSTRACT

Compositions, systems and methods for the detection of analytes with labeled nanostructures are provided. In particular, compositions and systems including labeled nanostructures for detecting a biomolecule of interest, and methods of use thereof, are provided.

### CROSS-REFERENCE TO RELATED APPLICATION

This application claims priority to copending U.S. provisional patent application Ser. No. 60/728,572, entitled “Detection of Biomolecules” filed on Oct. 20, 2005, which is entirely incorporated herein by reference.

### STATEMENT REGARDING FEDERALLY SPONSORSED RESEARCH OR DEVELOPMENT

This invention was made with government support under ECS 0404066 awarded by the National Science Foundation. The government has certain rights in the invention.

### FIELD OF THE INVENTION(S)

The present disclosure is generally directed to systems and methods for detection of analytes, in particular, the detection of biomolecules using nanostructures, particularly nanorods. The present disclosure is further directed to systems and methods for detection of cells containing a biomolecule of interest, such as a virus or other infectious agent.

### BACKGROUND

Respiratory syncytial virus (RSV) is a single-stranded, negative-sense RNA virus in the Paramyxovirus family that is the most important cause of serious lower respiratory tract illness (LRTI) in infants and young children worldwide, as well as an important pathogen in the elderly and immune compromised patient. RSV generally initiates mild upper respiratory tract infection in young children with infection rates approaching 50% in the first year of life. However, up to 40% of infected children develop serious lower respiratory tract disease with a substantial number of patients requiring hospitalization. RSV infection may cause respiratory failure in immune compromised patients with mortality rates of up to 70% in this population. RSV infection is associated with the clinical diagnosis of pneumonia and bronchiolitis, and RSV infection may predispose for asthma, or lead to otitis media.

There are two major groups of RSV, strains A and B, and both strains co-circulate. However, the clinical severity of RSV infection has not been conclusively linked with infection by either strain. Despite over four decades of research, no safe and effective RSV vaccine exists and treatments are limited. In infants and young children, exposure to RSV infection does not engender a protective immune response, as repeat infections with the same or different strains of RSV are common. These indications suggest that RSV may modulate or evade the immune response to promote virus infection, replication, and possibly virus persistence.

Consistent with this hypothesis, accumulating evidence in animal models and in cell lines suggests that RSV may cause latent or persistent infection; however, the power of these results has been limited by the lack of sensitivity of virus detection. The significant public health burden mediated by RSV infection is exemplified by the dramatic infection rate in younger children, the percent of children hospitalized because of RSV-associated LRTI, and by the substantial mortality in the young and immune compromised patient.

Commercial rapid RSV detection kits exist to support critical anti-viral therapy recommendations (e.g., BD Directigen™ RSV Test and Abbott TestPack RSV™). However, these kits have limited sensitivity, and a lack of specificity in some patients requires confirmation by additional tests to rule out false-positive results and/or detection of other respiratory viruses.

The current state-of-the-art for viral diagnostic methods involves isolation and cultivation of viruses and may employ (1) an enzyme-linked immuno-sorbant assay (ELISA), a method that uses antibodies linked to an enzyme whose activity can be used for quantitative determination of the antigen with which it reacts, or (2) polymerase chain reaction (PCR), a method of amplifying fragments of genetic material so that they can be detected. These diagnostic methods are cumbersome, time-consuming, sometimes unreliable, and ELISA has limited sensitivity.

For RSV in particular, isolation of the virus in cell culture has been considered the reference diagnostic method, followed by immunofluorescence assay (IFA) or enzyme immuno-sorbant assay (EIA). However, results from virus isolation studies are not rapidly available for patient management, and are not sufficiently sensitive to detect infection in a substantial portion of patients. There is, therefore, a critical need for a rapid, reproducible and highly sensitive and specific method of diagnosing viruses such as RSV that inflict substantial disease burdens on human and animal health and for other respiratory viruses that also pose a significant threat as agents for bioterrorism. The emergence of biosensing strategies that leverage nanotechnology for direct, rapid, and increased sensitivity in detection of viruses, both for public health and homeland security applications, are needed to bridge the gap between the unacceptably low sensitivity levels of current bioassays and the burgeoning need for more rapid and sensitive detection of infectious agents and other biomolecules.

### SUMMARY

Briefly described, the present disclosure provides compositions, systems and methods of detecting an analyte of interest (e.g., a biomolecule) in a sample. Compositions of the present disclosure include a plurality of nanostructures, in particular, nanorods (including heterostructured nanorods made of more than one material), where the nanorods include a binding agent having an affinity for the biomolecule of interest coupled to the surface of the nanorod and a reporter molecule coupled to the surface of the nanorod, where the reporter molecule is capable of providing a detectable signal. Embodiments of systems of the present disclosure include the nanorod compositions of the present disclosure and a detecting device for detecting the presence of the labeled nanorods in a sample.

In embodiments, the biomolecule of interest to be detected is selected from one of the following: polypeptide, protein, glycoprotein, nucleic acid, carbohydrate, lipid, vitamin, virus, a virus infected cell, and combinations thereof. In particular embodiments, the biomolecule is a virus or virus-infected cell. In embodiments, the binding agent is selected from: polynucleotide, polypeptide, protein, glycoprotein, lipid, carbohydrate, fatty acid, fatty esters, macromolecular polypeptide complex, and a combination thereof. In particular, the binding agent is an antibody or fragment thereof.

Methods of the present disclosure include attaching at least one binding agent to an array of labeled nanorods on a substrate, removing the nanorods from the substrate to provide a composition of labeled nanorods, contacting the composition of labeled nanorods with the sample containing the analyte of interest (e.g., a second biomolecule), and detecting the presence of the labeled nanorods. In an embodiment, a method for detecting a biomolecule of interest in a sample includes contacting the sample with a composition comprising a plurality of labeled nanorods including a binding agent having an affinity for the biomolecule of interest, where the labeled nanorods are capable of providing a detectable signal and, in the presence of the biomolecule of interest, bind the biomolecule of interest; and detecting the signal produced by the labeled nanorods to determine the presence or absence of the biomolecule of interest.

Embodiments of methods of making the labeled nanorods and nanorod compositions of the present disclosure include the following steps: providing a substrate; depositing an array of nanorods on the substrate (e.g., by galancing angle vapor deposition); labeling the nanorods by immobilizing a reporter molecule onto at least a portion of the surface of each nanorod; immobilizing a binding agent having an affinity for the biomolecule of interest onto a portion of the surface of each nanorod; and removing the nanorods from the substrate to form a composition of nanorods.

Other aspects, compositions, methods, features, and advantages of the present disclosure will be or become apparent to one with skill in the art upon examination of the following drawings and detailed description. It is intended that all such additional compositions, methods, features, and advantages be included within this description, be within the scope of the present disclosure, and be protected by the accompanying claims.

### BRIEF DESCRITPION OF THE FIGURES

The disclosure can be better understood with reference to the following drawings. The components in the drawings are not necessarily to scale, emphasis instead being placed upon clearly illustrating the principles of the present disclosure.

FIG. 1A illustrates an embodiment of a method of making the labeled nanorods of the present disclosure. FIG. 1 B illustrates an embodiment of a method of using the labeled nanorods of a present disclosure to detect a virus-infected cell.

FIG. 2 illustrates embodiments of modified oblique angle deposition (OAD) and glancing angle deposition systems for a planar substrate (e.g., a chip or glass slide).

FIG. 3 shows SEM images taken from a top view (left) and a cross section (right) of a sample of Si nanorods formed on a substrate.

FIG. 4 shows TEM images of individual Si nanorods in solution after removal from the substrate.

FIG. 5 shows TEM images of gold coated Si nanorods in solution after removal from the substrate.

FIG. 6 shows SEM images taken from a top view (left) and a cross section (right) of FITC and Au coated nanorods on a substrate.

FIG. 7 shows TEM images of the FITC and Au coated nanorods in solution after removal from the substrate.

FIG. 8A is a graph illustrating a UV-Vis spectrum of Si nanorods before and after annealing and dye (FITC) immobilization. FIG. 8B illustrates a UV-Vis spectrum of gold-coated Si nanorods before and after annealing and dye (FITC) immobilization.

FIG. 9A is a Typhoon scan image of dye-conjugated Si nanorods on a substrate. FIG. 9B is a Typhoon scan image of Si nanorods on a substrate without dye molecules.

FIG. 10 shows Typhoon scan images of FITC conjugated Si nanorods on a substrate (right) and an FITC conjugated Si film (left).

FIG. 11 shows digital or scanned confocal microscope images of FITC conjugated Si nanorods (FIG. 11A) and FITC conjugated Si film (FIG. 11B).

FIG. 12 shows Typhoon scan images of a glass slide including four droplets of nanorod suspensions and a control: (A) FITC conjugated Si nanorods; (B) FITC conjugated, Au coated 15 nm Si nanorods; (C) FITC conjugated, Ag coated Si nanorods; (D) FITC conjugated, Au coated 7.5 nm Si nanorods, and (E) deionized water.

FIGS. 13A-13D are scanned images showing detection of RSV infected cells with a composition of antibody conjugated Au/FITC/Si nanorods. FIG. 13A illustrates a sample of RSV infected cells with a ¼ dilution of the nanorod solution; FIG. 1 3B illustrates a sample of non-infected cells with a ¼ dilution of the nanorod solution; FIG. 13C illustrates a sample of RSV infected cells with a ½ dilution of the nanorod solution; and FIG. 13D illustrates a sample of non-infected cells with a ½ dilution of the nanorod solution.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere. Experimental hypoxia was obtained by growing cells in culture medium in an incubator under an environment of 1% partial pressure of oxygen unless otherwise indicated.

Embodiments of the present disclosure will employ, unless otherwise indicated, techniques of synthetic organic chemistry, biochemistry, molecular biology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Prior to describing the various embodiments, the following definitions are provided and should be used unless otherwise indicated.

Definitions:

The term “nucleic acid” or “polynucleotide” is a term that generally refers to a string of at least two base-sugar-phosphate combinations. As used herein, the term includes deoxyribonucleic acid (DNA) and ribonucleic acid (RNA) and generally refers to any polyribonucleotide or polydeoxyribonucleotide, which may be unmodified RNA or DNA or modified RNA or DNA. RNA may be in the form of an tRNA (transfer RNA), snRNA (small nuclear RNA), rRNA (ribosomal RNA), mRNA (messenger RNA), anti-sense RNA, RNAi, siRNA, and ribozymes. Thus, for instance, polynucleotides as used herein refers to, among others, single- and double-stranded DNA, DNA that is a mixture of single-and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid sequence” or “oligonucleotide” also encompasses a nucleic acid or polynucleotide as defined above.

It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia.

For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein.

The term also includes PNAs (peptide nucleic acids), phosphorothioates, and other variants of the phosphate backbone of native nucleic acids. Natural nucleic acids have a phosphate backbone; artificial nucleic acids may contain other types of backbones, but contain the same bases. Thus, DNAs or RNAs with backbones modified for stability or for other reasons are “nucleic acids” or “polynucleotides” as that term is intended herein.

Use of the phrase “biomolecule” is intended to encompass deoxyribonucleic acid (DNA), ribonucleic acid (RNA), nucleotides, oligonucleotides, nucleosides, proteins, peptides, polypeptides, selenoproteins, antibodies, antigents, protein complexes, viruses and other molecular pathogens and toxins, combinations thereof, and the like. In particular, the biomolecule can include, but is not limited to, naturally occurring substances such as polypeptides, polynucleotides, lipids, fatty acids, glycoproteins, carbohydrates, fatty acids, fatty esters, macromolecular polypeptide complexes, vitamins, co-factors, microorganisms such as viruses, bacteria, protozoa, archaea, fungi, algae, spores, apicomplexan, trematodes, nematodes, mycoplasma, or combinations thereof, as well as cells (e.g., eukaryotic cells and prokaryotic cells) infected with viruses, toxins, and/or other molecular pathogens.

In a preferred aspect, the biomolecule is a virus, including, but not limited to, RNA and DNA viruses. In particular the biomolecule is a virus, which may include, but is not limited to, a retrovirus (e.g., human immunodeficiency virus (HIV), a feline immunodeficiency virus (FIV), a simian immunodeficiency virus (SIV), a porcine immunodeficiency virus (PIV), a feline leukemia virus, a bovine immunodeficiency virus, a bovine leukemia virus, a equine infectious anemia virus, a human T-cell leukemia virus), a Pneumovirus (e.g., respiratory syncytial virus (RSV)), Paramyxoviridae (e.g., Paramyxovirus (Parainfluenzavirus 1-4, Sendai virus, mumps, Newcastle disease virus)), a Metapneumovirus (e.g., human and avian metapneumovirus), and Orthomyxoviridae (e.g., an influenza virus A, B, C). In addition, the biomolecule may include additional viruses including, but not limited to, an astrovirideae, a calivirideae, a herpes virus, a picornaviridea, a poxuvirideae, a reovirideae, a togavirideae, an avian influenza virus, a polyomavirus, an adenovirus, a rhinovirus, a Bunyavirus, a Lassa fever virus, an Ebola virus, a corona virus, an arenavirus, a Filovirus, a rhabdovirus, an alphavirus, a flavivirus, Epstein-Barr Virus (EBV), and viruses of agricultural relevance such as the Tomato Spotted Wilt Virus.

In another exemplary embodiment, the biomolecule is a surface molecule or surface antigen on the surface of a pathogen (e.g., a bacterial cell, a spore, etc.), or the biomolecule is a toxin or other byproduct of a pathogen (e.g., a toxin produced by a bacterial cell). Other examples of biomolecules are viral projections such as Hemmaglutinin and Neuraminidase.

Use of the phrase “peptides”, “polypeptide”, or “protein” is intended to encompass a protein, a glycoprotein, a polypeptide, a peptide, fragments thereof and the like, whether isolated from nature, of viral, bacterial, plant, or animal (e.g., mammalian, such as human) origin, or synthetic, and fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

Use of the term “affinity” can include biological interactions and/or chemical interactions. The biological interactions can include, but are not limited to, bonding or hybridization among one or more biological functional groups located on the binding agent and the biomolecule of interest. In this regard, the binding agent can include one or more biological functional groups that selectively interact with one or more biological functional groups of the biomolecule of interest. The chemical interaction can include, but is not limited to, bonding (e.g., covolent bonding, ionic bonding, and the like) among one or more functional groups (e.g., organic and/or inorganic functional groups) located on the biomolecule of interest and binding agent.

Discussion:

Generally described, the present disclosure provides methods and systems for the detection, analysis, and/or quantification of an analyte (e.g., a biomolecule). One aspect, among others, provides methods and systems for the detection of a biomolecule using labeled nanostructures. In particular, the system and methods can be used to determine the presence, qualitatively and/or quantitatively, of one or more types of biomolecules (e.g., viruses), cells (e.g., virus infected cells), spores, toxins, drugs, contaminants, biohazards, and other chemical agents of interest (e.g. biochemical agents, explosives, nuclear wastes). For clarity, this disclosure describes the use of the system with biomolecules, but one skilled in the art would understand that the system can be used to determine the presence of other targets of interest, such as those described above, to which a complimentary binding agent exists or can be designed.

The nanostructures can include, but are not limited to, nanorods, nanospheres, nanowires, nanotubes, nanospirals, combinations thereof, and the like. For clarity, this disclosure describes the use of the system with nanorods, but one skilled in the art would understand that the compositions, systems, and methods of the present disclosure can be used with other nanostructures such as, but not necessarily limited to, those listed above. In an exemplary embodiment, the nanostructure is functionalized with one or more binding agent(s) having an affinity for an analyte of interest. The binding agent is capable of binding (e.g., ionically covalently, hydrogen binding, and the like) or otherwise associating with (e.g., chemically, biologically, etc.) one or more biomolecule(s) or other analyte of interest. The nanostructure is also preferably labeled with a reporter molecule (e.g., a fluorescent or luminescent dye) to allow detection of the bound nanostructure in a sample being tested for the presence of a biomolecule.

In some exemplary embodiments the biomolecule, as defined above, to be detected includes, but is not limited to, viruses, and biological molecules such as, polypeptides, polynucleotides, lipids, fatty acids, carbohydrates, vitamins, co-factors, and combinations thereof. In some particular embodiments of the present disclosure, the biomolecule to be detected is within a cell, thus allowing the detection of cells infected with, or otherwise harboring, a virus, toxin, or other biomolecule of interest. In a preferred aspect, the biomolecule is a virus, for example a respiratory syncytial virus. In another embodiment, the biomolecule is a surface molecule or surface antigen on the surface of a pathogen (e.g., a bacterial cell), or the biomolecule is a toxin or other byproduct of a pathogen (e.g., a toxin produced by a bacterial cell). Other examples of biomolecules are viral projections such as Hemmaglutinin and Neuraminidase.

The binding agent can also be a biomolecule, such as, but not limited to, a polynucleotide, a polypeptide, a carbohydrate, a lipid, or the like. Exemplary polypeptide binding agents include, but are not limited to, antibodies or fragments thereof and antigens or fragments thereof. The binding agent can be attached to a surface of the nanostructure using conventional linking chemistry. When a biomolecule is introduced to the nanostructure, the biomolecule binds or otherwise interacts with the binding agent bound to the nanostructure. Generally, the biomolecule can be present or believed to be present in a cell, tissue, or fluid sample. Exemplary samples include buccal cells, buffered solutions, saliva, sweat, tears, phlegm, urine, blood, plasma, cerebrospinal fluid, or combinations thereof. Because the nanostructure is labeled, interaction between the biomolecule and the binding agent can be detected, for example via fluorescence or another signal that can be detected. In exemplary embodiments, the signal is provided by a reporter molecule, such as a fluorescent dye molecule, bound to or otherwise associated with the nanostructure.

Embodiments of the present disclosure also relate to methods of using the nanorod detection system to detect biomolecules in a sample, and methods of fabricating the nanorods. The nanorod detection system can enhance the detection of molecules (e.g., biomolecules) by a number of orders of magnitude (e.g., about 1 to 3 orders of magnitude) in a reproducible, specific, and accurate manner.

In general, the nanorod detection system includes, but is not limited to, a plurality of nanostructures (e.g., nanorods) in a composition, such as a solution, suspension, gel, colloid, sol, or the like, which can be applied to a sample to detect a biomolecule of interest. In embodiments, the nanorods are labeled with a reporting molecule capable of producing a detectable signal. Reporter molecules for use in the present disclosure include any substance capable of being coupled to the nanostructure and capable of producing a detectable signal, such as, but not limited to, molecules with particular optical, electrical, and magnetic properties that can generate a distinguishable signals different from the detecting target, such as, for instance, fluorescent dyes and fluorescent quantum dots.

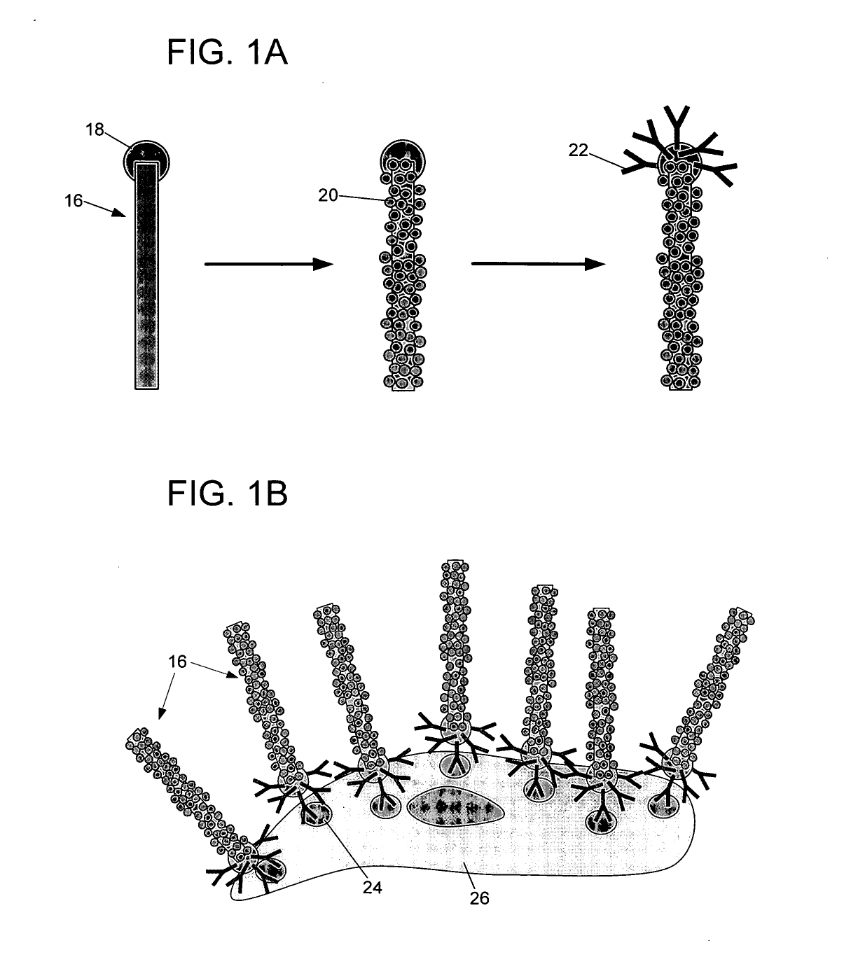

As illustrated schematically in FIG. 1A, the nanorods 16, having a metal-coated tip 18, are labeled with a reporting molecule 20 capable of producing a detectable signal (e.g., a fluorescent or luminescent dye) and are functionalized with a binding agent 22 (e.g., an antibody) capable of binding or otherwise associating with the biomolecule of interest (e.g., an antigen, such as a virus). Thus, when the composition of nanorods is applied to a sample to be tested (e.g. cells obtained from a host), if the biomolecule is present, the nanorods 16 associate with the biomolecule via the binding agent 22, and the presence can be detected via the signal (e.g., fluorescence) provided by the reporting molecule 20. As illustrated in FIG. 1B, the labeled and functionalized nanorods 16 are applied to a sample containing cells 26, and the nanorods associate with the analyte of interest 24 within or on the surface of the cell (e.g., virus particles within or expressed on the surface of an infected cell). Further, the high aspect ratio of the nanorods provides significant signal enhancement (e.g., 40 times) over conventional detection systems. The aspect ratio of the nanorods can be controlled (e.g., increased, by varying factors such as the size and shape of the nanorods, as well as the materials used to fabricate the nanorods.

The nanostructures can include, but are not limited to, nanorods, nanowires, nanotubes, nanospirals, combinations thereof, and the like. In exemplary embodiments, the nanostructures are nanorods. The nanostructures (e.g., nanorods) can be fabricated of one or multiple (e.g., two or more) materials including, but not limited to, silicon and/or an oxide thereof, a metal, a metal oxide, a metal nitride, a metal oxynitride, a compound, a doped material, a polymer, a multicomponent compound, and combinations thereof. In exemplary embodiments, the nanorods are heterogenous (e.g., formed from two or more different materials). The metals can include, but are not limited to, silver, nickel, aluminum, silicon, gold, platinum, palladium, titanium, copper, cobalt, zinc, other transition metals, composites thereof, oxides of each, nitrides of each, silicides of each, phosphides (P³⁻) of each, oxynitrides of each, and combinations thereof. In particular, the materials can include the following: silicon, silicon oxide, germanium, gold, silver, nickel, and titanium oxide. The composition of the nanorods is the same as that of the materials described herein or a combination of the materials described herein, or alternative layers of each. In preferred embodiments, the nanorods are made of a biocompatible material. In an exemplary embodiment, the nanorods are made of silicon with a metal layer provided over at least part of the silicon nanorod, to form a heterogeneous nanorod. Such a metal layer may include any of the materials described above for the nanorods, or a ceramic material having electrical conductivity similar to that of metals. In particular the metal layer is gold or silver.

The length/height of the nanorod can be from a few hundred nanometers to over a few thousand nanometers. In particular, the nanorods can have a height from about 100, 200, 300, 400, 500, 600, 700, 800, and 900 nanometers to about 500, 600, 700, 800, 900, 1000, 1100, 1200, 1300, 1400, and 1500 nanometers to about 2000, 2500, 3000 nanometers. The length depends, at least in part, upon the deposition time, deposition rate, and the total amount of evaporating materials. When formed on the substrate, the substrate can have nanorods of the same height or of varying heights on one or more portions of the substrate. In particular, the nanorods have a height of about 100 to 1500 nanometers.

The diameter is the dimension perpendicular to the length. The diameter of the nanostructure is about 10 to 30 nm, about 10 to 60 nm, about 10 to 100 nm, and about 10 to 150 nm. In embodiments the nanorods can have a diameter of about 50 to 120 nanometers; preferably, they have a diameter of about 50 to 100 nm. One or more of the dimensions of the nanostructure could be controlled by the deposition conditions and the materials. The substrate can have from tens to tens of thousands or more nanorods formed on the substrate. The number of nanorods, height, and diameter of the nanorods, and composition of the nanorods depend upon the specific application of the nanorod detection system and can be tailored accordingly by one of skill in the art.

A method of making the nanorods of the present disclosure includes providing a planar (or “flat”) substrate (such as a silicon, quartz, or glass substrate) and depositing the nanorods on the substrate. Planar substrates may also be made of materials including, but not limited to, semiconductors (e.g., Si, GaAs, GaAsP, and Ge), oxides (e.g., SiO₂, Al₂O₃), and polymers (e.g., polystyrene, polyacetylene, polyethylene, etc.). The nanorods may be deposited on the substrate by many methods known to those of skill in the art, such as, but not limited to, chemical vapor deposition, sputtering growth, electrochemical deposition, glancing angle deposition, or a combination of two or more different deposition methods. In an exemplary embodiment, the nanorods are depostited by glancing angle deposition. Methods of depositing nanorods on a planar substrate by glancing angle deposition are described in greater detail in U.S. patent application Ser. No.: 11/376,661 entitled “Surface Enhanced Raman Spectroscopy (SERS) Systems, Substrates, Fabrication Thereof, And Methods of Use Thereof ” filed Mar. 15, 2006, and incorporated herein by reference in its entirety. Such methods are described briefly below with respect to some exemplary embodiments.

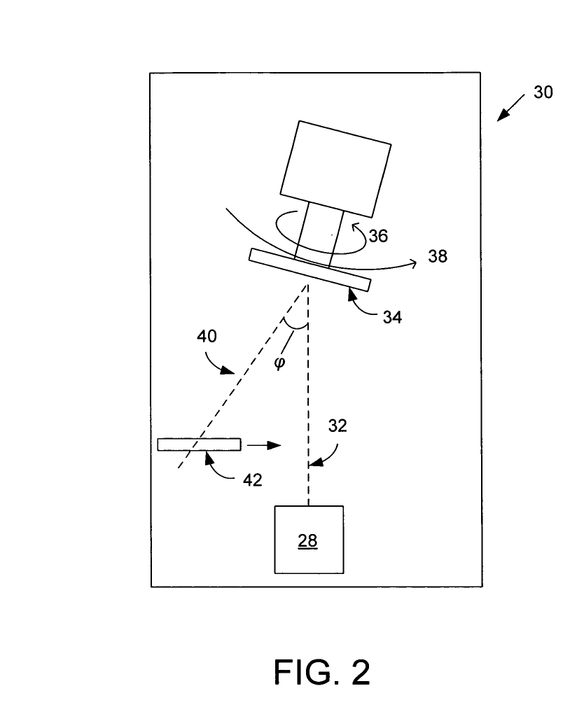

An embodiment of a modified oblique angle deposition (OAD) system for glancing angle deposition of nanorods on a planar substrate is illustrated in FIG. 2. The embodiment of an OAD system 30 illustrated in FIG. 2 includes, but is not limited to, an evaporation source 28, a substrate 34, a shutter 42, and a substrate manipulation mechanism (e.g., one or more motors) to move (e.g., rotate) the substrate relative to the evaporation source 28. The system 30 includes a motor for moving the planar substrate 34 in a polar rotation 38, which changes the incident angle (φ) between the surface normal axis of the substrate (e.g., axis 40) and the vapor source direction (e.g., vapor arrival line 32). In some embodiments, the system 30 may optionally include a second motor for rotating the planar substrate in an azimuthal rotation 36, which allows additional control over the shape of the nanorods. In embodiments of the deposition system 30 the evaporation device 28 can include a physical vapor deposition (PVD) system, such as thermal evaporation, e-beam evaporation, molecular beam epitaxy (MBE), sputtering growth, pulsed laser deposition, combinations thereof, and the like. To deposit the nanorods on the the planar substrate 34, the substrate is then rotated polarly in order to make an incident angle φ less than about 89° (e.g., where φ is from about 75° to 89°, about 80° to 86°, and about 86°), of the surface normal of the substrate with respect to the incoming vapor direction. This process is described in greater detail in patent application Ser. No.11/376,661, and will not be described further herein.

In an exemplary embodiment, the nanorods are made of silicon and are made by exposing a first portion of a substrate to a silicon vapor by opening a shutter 42 to a first setting. The first setting exposes a predetermined portion of the substrate. A first nanorod at a first position on the substrate is formed. The first nanorod grows to a first height (e.g., about 200 nanometers). Subsequently, the shutter is opened to a second setting, thereby exposing the first portion and a second portion to the silicon vapor. A second nanorod is formed at a second position on the substrate. The second nanorod grows to the first height (e.g., 200 nanometers). In this step the first nanorod grows to a second height (e.g., 400 nanometers), where the second height is about twice as high as the first height. This process can be repeated to expose a plurality of portions on the substrate to create a plurality of nanorods of various lengths. For example, nanorods of the following lengths can be prepared: about 200 nanometers, about 400 nanometers, about 600 nanometers, about 800 nanometers, about 1000 nanometers, and about 1500 nanometers.

In particular, the nanorods can be formed using glancing angle vapor deposition, as described above. In one embodiment, the incident angle is from about 75° to 88°. In an exemplary embodiment, a layer of thin metal film (e.g., gold or silver) is coated onto the Si nanorods directly via sputtering growth or thermal evaporation, which methods are known to those of skill in the art. In an exemplary embodiment, a layer of metal (e.g., gold or silver) is applied to the nanorods by sputtering with a tilting angle of about 30°. The thickness of the metal layer is generally between about 20 nm and 100 nm.

After formation of the nanorods, the reporter molecule and binding agent are selectively immobilized on the surface of the nanorods using conventional linking chemistry (e.g., biologically (e.g., hybridization) and/or chemically (e.g., ionically or covalently)). For instance, the nanorods can be labeled and/or functionalized with the binding agent by immobilizing the reporter molecules (e.g., fluorescent dye molecules) and/or the binding agent (e.g., an antibody) on the nanorod surface by annealing to the metal (e.g., Si, Ag, or Au) surface of the nanorod via a linking agent (e.g., DSP (dithiobis(succinimidyl propionate)) or SAM (self-assembly monolayer)). In an embodiment the nanorods and substrate can be annealed at about 400° C. under oxygen atmosphere in order to oxidize the surface of the nanorods. Preferably, the nanostructures are labeled with the reporter molecules and functionalized with the binding agent while still on the substrate to help prevent aggregation of the nanorods in a solution.

In certain aspects, a fluorescent dye is used as the reporter molecule to label the nanorods, and the binding agent is an antibody. In a preferred embodiment, the surface of the nanorods is oxidized to aid in binding the dye and/or antibody. In one embodiment, the dye molecules are annealed by attachment between the dye ester and 3-aminopropyltriethoxysilane (APTES) on the oxidized Si nanorods. The ethoxy group of the APTES undergoes a displacement reaction with SiOH groups on the silicon oxide, resulting in an —NH2 surface termination, which provides the ability to bind the dye molecules. Suitable dye molecules include, but are not limited to, Alexa 350, Alexa 430, Alexa 488, Alexa 532, Alexa 546, Alexa 568, and Alexa 594 dyes, AMCA, Lucifer Yellow, fluorescein, luciferins, aequorins, rhodamine 6G, tetramethylrhodamine or Cy3, lissamine rhodamine B, and Texas Red, respectively (the numbers in the Alexa names indicate the approximate excitation wavelength maximum in nm).

In embodiments, the nanorods are functionalized by immobilizing the binding agent (e.g., an antibody) on the nanorod surface by annealing to the metal (e.g, Au or Ag) surface of the nanrod via a linking agent (e.g., DSP (dithiobis(succinimidyl propionate)) SAM (self-assembly monolayer)). In embodiments, the reporter molecule is coupled to the silicon portion of the nanorod and the binding agent is coupled to the portion of the nanorod having a metal layer deposited thereon. Additional details regarding the methods of making the nanorods of the present disclosure can be found in the examples below.

Once the nanorods are formed, labeled, and functionalized on the substrate, they are removed from the substrate and dispersed into a solvent to form a nanorod composition to be used to detect a biomolecule of interest in a sample. Such composition may be in the form of a solution, a suspension, a gel, a sol, a colloid, or the like. The nanorods may be removed from the substrate via any method that can preserve the reactivity of the nanorods after removal. Exemplary methods include sonication, mechanical ablation, selective chemical desorption, etc. In alternative embodiments, the nanorod may be first removed from the substrate and then modified as above (e.g., labeled and functionalized with a binding agent), leading to the same resulting nanorod composition.

Once the nanorods are free from the substrate, they can detect biomolecules in a great variety of samples. Since the binding agent on the nanorods has an affinity for a target biomolecule, when the nanorods are introduced to a sample containing the analyte of interest, such as a biomolecule, the biomolecule binds or otherwise interacts with the binding agent bound to the nanostructure. Unbound nanorods can then be washed or otherwise removed from the sample, and the presence of bound nanorods (indicating the presence of the analyte of interest) can be detected by the signal produced by the reporter molecule. Generally, the analyte/biomolecule can be present or believed to be present in a sample, such as a gaseous, tissue or fluid sample. For instance, the nanorods can detect biomolecules (such as a virus or molecular toxin) present in living cells, due to the ability for individual nanorods to enter a cell to associate with biomolecules within a cell, or to associated with biomolecule presented on the surface of the cell, such as virus antigens. In this way, the nanorod probes are able to detect cells infected with a specific pathogen (as illustrated in FIG. 1).

In embodiments the methods and systems of the present disclosure can be used for enhanced detection and quantification of an analyte of interest, as described briefly below. The fluorescent intensity I in general is proportional to the number N of dye molecules in a illuminated area: I∝N. For a microscopic surface area ΔA on a cell surface, the total number of dyes that can be conjugated to that area is
N_{c}=ΔA/Aₘ, (1)
where Aₘ is the area occupied by a single dye conjugates molecules.

If nanorods are used for the detection instead of conjugated dye molecules, then the total number of nanorods on the area ΔA will be
Nᵣ=ΔA/A_{R} (2)
where Aᵣ is the cross-section area of the nanorods. For each nanorod, assuming its length l, radius r, then the total exposed area of a nanorod is
Aₑ=πr²+2πrl, Aᵣ=πr² (3)

The total number of dye on a nanorod is
N_{dr}=(πr²+2πrl)/Aₘ (4)

Then the total number of dye on area ΔA is

$${N}_{t} = {N}_{r} {N}_{dr} = \frac{Δ A}{π {r}^{2}} \frac{π {r}^{2} + 2 π rl}{{A}_{m}} = {N}_{c} ( 1 + \frac{2 l}{r} ) \qquad ( 5 )$$

Therefore, the aspect ratio of a nanorod determines the signal enhancement. In an embodiment where the nanorods had the following general dimensions, l˜1000 nm, 2r˜100 nm, the enhancement is about 40-fold.

Due to the unique structure of the nanorods, as well as their unique chemical properties, they may be used in many applications in addition to those specifically discussed herein. For instance, they can be used to develop multiplexing detection or imaging assays by employing different dye molecules with different colors immobilized onto nanorods with binding agents specific for different analytes of interest, where a specific dye corresponds to a nanorod including a binding agent for a specific analyte, so that more than one analyte can be detected in a sample in a single assay. In this manner, one can use the color image to simultaneously recognize cells infected by different viruses or different pathogens or other biomolecules presented in the same samples. The nanorods can also be used for targeted drug delivery. For instance, drug molecules specifically targeted to a certain virus can be immobilized onto Si nanorods, and an antibody specific to the same virus can be immobilized onto the Au tips. Thereby an entity of targeted drug is formed to specifically target a particular virus/pathogen in a sample or a host. Similarly, specific genes can be immobilized onto Si nanorods for targeted delivery purposes.

Additionally, the properties of the nanorods may be varied to provide other characteristics and applications. For instance, the signal enhancement provided by the nanorods may be further increased by using high aspect ratio metallic nanorods, this includes using the principle of enhanced fluorescence, enhanced inferred spectroscopy, surface enhanced Raman spectroscopy, surface plasmon resonance, and the like. Replacing the Si nanorod with metallic nanorod introduces high local electric field for fluorescence enhancement due to the electromagnetic effect. This can bring at least another 2-3 orders of magnitude of enhancement. Additionally, reducing the nanorod diameter can also increase the aspect ratio. In a further aspect, using magnetic rods, or IR active rods, as the nanorods of the present disclosure, provides the ability for localized tumor and cancer therapy.

It should be emphasized that the above-described embodiments of the present disclosure, particularly, any “preferred” embodiments, are merely possible examples of implementations, and are merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

### EXAMPLES

### Example 1

### Nanorod Fabrication

Materials and Methods

The Si nanorods were fabricated by glancing angle deposition method. The basic deposition setup is shown in FIG. 2. The collimated evaporation beam has a large incident angle (p with respect to the substrate surface normal. The substrate is manipulated by two stepper motors: one motor controls the incident angle (p and another motor controls the azimuthal rotation of the substrate with respect to substrate surface normal. During the deposition, the substrate is rotated azimuthally at a fixed incident angle (86°); the deposition rate (4 Å/s) was monitored by the quartz crystal microbalance, while the rotation speed (50-100 Hz) was controlled by a computer. By fixing the incident angle at relatively glancing and continuously rotating the substrate azimuthally with a suitable constant speed, vertically aligned Si nanorods were formed. The experiments were performed in a high vacuum chamber with a background pressure of 1×10⁻⁷ Torr. The pressure during deposition was less than 1×10⁻⁵ Torr. The Si (99.9995%, from Alfa Aeser) was evaporated by the e-beam evaporation method.

SEM and TEM Images

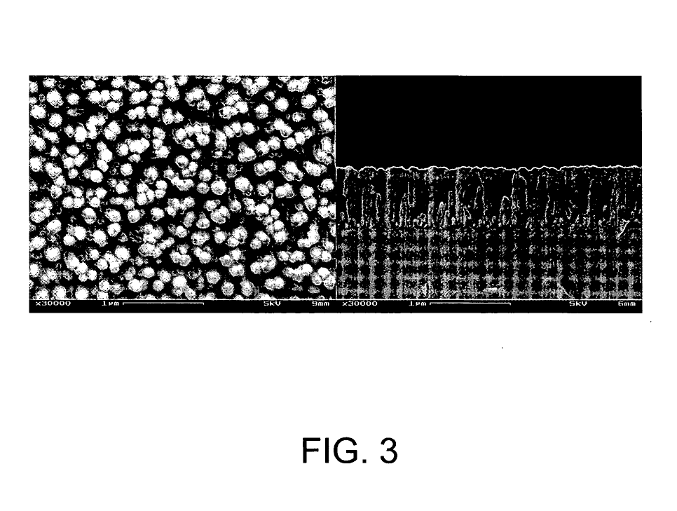

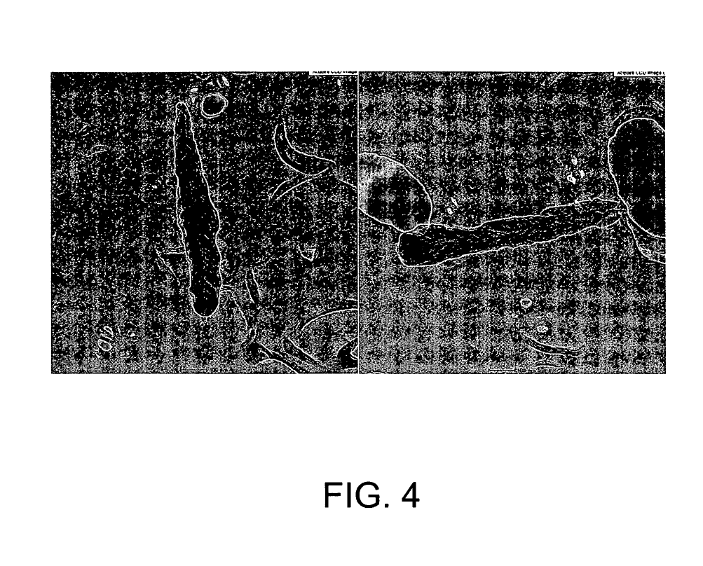

Si nanorod samples were formed with normal film thicknesses of 2000 nm. The real nanorod height was determined by a field emitting scanning electron microscope (SEM: LEO 982). FIG. 3 shows the top-view and cross-section SEM images of the Si nanorod sample with 2000 nm normal film thickness. Si nanorods were detached from the substrate into DI water by sonication. Some representative TEM images of individual Si nanorods are shown in FIG. 4.

The use of nanorods provides a larger surface area compared with a flat detection surface. Assuming the nanorod has a cylindrical shape the estimated dimensions are approximately the following:

| Diameter | | | |
|---|---|---|---|
| (nm) | Length (nm) | Density (number/um²) | Surface area (um²) |
| 150 | 900 | 20 | 8.48 |

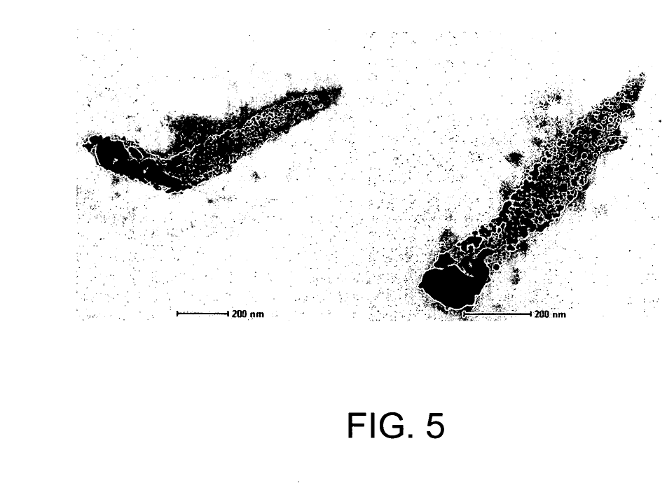

The surface area ratio of the nanorods surface and the film surface is about 8.48. Gold (SPI-MODULE™ Sputter Coater, 100% Au) was sputtered on the top of some of the Si nanorods by different angles while the Si nanorods still stand on the substrate. As shown in TEM images (FIG. 5 ), the black parts indicate that the gold has been sputtered on the Si nanorods.

Si nanorods chips prepared as described above were immersed in 2% (3-Aminopropyl)triethoxysilane (APTES, ≧98%, Sigma-Aldrich)/acetone overnight at 45° C. Alexa 488-succinimide ester, fluorescein-5-isothiocyanate (FITC ‘Isomer I’) or dansyl-X SE (Molecular Probes) was spread onto the APTES treated Si nanorods or Si film via the primary amine group. Excess dye was washed off by DI water and dried in nitrogen.

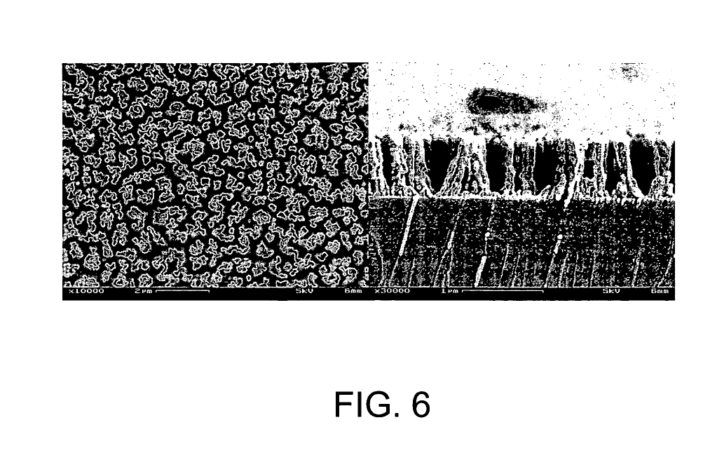

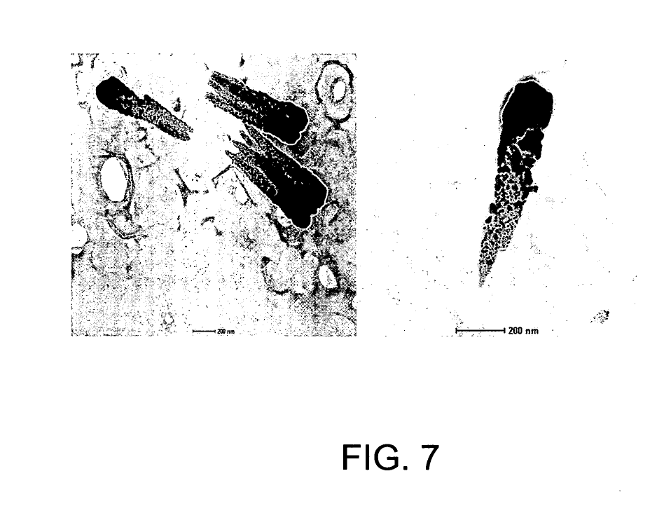

FIG. 6 shows top-view and cross-section SEM images of the FITC/Au/Si nanorods. The gold layer with a thickness of about 15nm was sputtered on the Si nanorods with 2000 nm normal film thickness in the normal direction. After the dye treatment, some nanorods bundled together as shown in FIG. 6. FIG. 7 shows TEM images of the nanorods after removal from the substrate via sonification. Bundling of the nanorods also observed after removal from the substrate, as shown in the TEM images (FIG. 7). The top and part of the sides of the Si nanorods were covered by gold.

UV-Vis Spectrum Measurements

The Si nanorods or Au(15 nm)/Si nanorods(2 μm, 86°) were deposited on a glass substrate for transparency. UV-Vis spectrum measurements were taken of the nanorods before and after the annealing of the nanorods for the silicon oxidization and the dye immobilization.

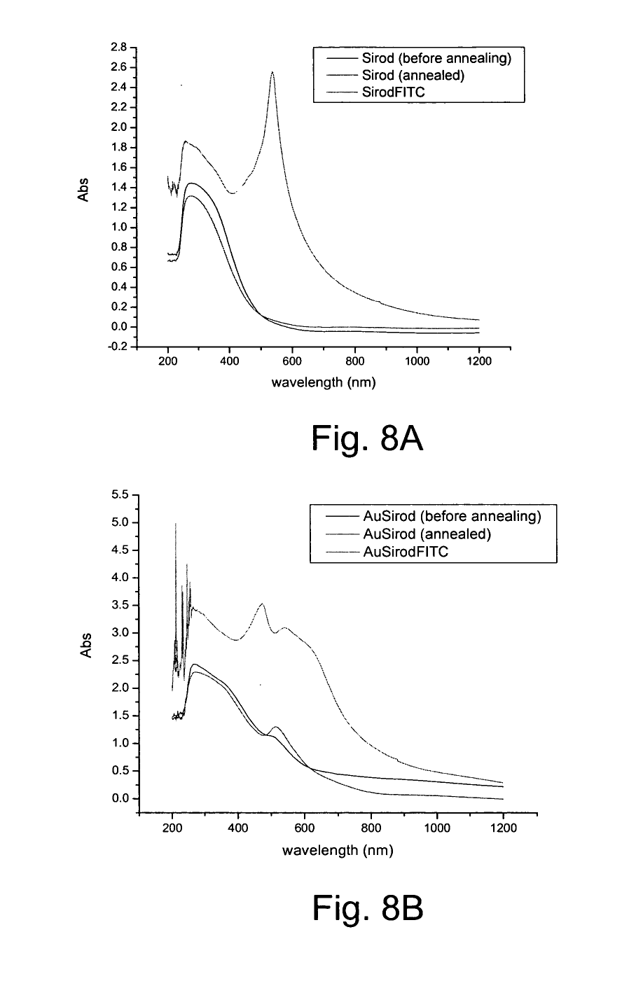

As illustrated in FIG. 8A, the peak around 280 nm represents the silicon. The intensity decreased after annealing because the surface of the Si nanorods has been oxidized into silica. The peak around 540 nm represents the FITC.

As illustrated in FIG. 8B, the peak at 520 nm is from the gold coating, while the FITC peak was slightly blue-shifted. The UV-Vis spectrum indicated that the coating was successfully coated onto the Si nanorods and that the dye was successfully conjugated to the silica surface of the nanorods.

Results

Dye-Conjugated Si Nanorods vs. Si Nanorods

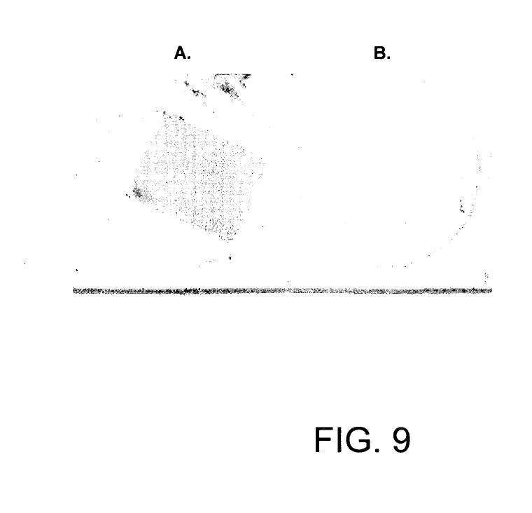

While still immobilized on the substrate, treated Si nanorods were scanned in the Typhoon scanner alongside a control substrate that had not been treated with dye. The image (FIG. 9) shows that the dye has been conjugated onto the APTES-treated Si nanorods successfully.

Dye-Conjugated Si Nanorods vs. Dye-Conjugated Si Film

As described in above, the dye dansyl-X SE was applied to both an APTES-treated Si film and the Si nanorods. Due to the larger surface area of the nanorods, the fluorescence microscope image of the dansyl-X SE conjugated Si nanorods was brighter than that of the dansyl-X SE conjugated Si film (data not shown).

FITC Conjugation

Activated dyes such as Alexa-488 ester can be applied to the APTES-treated Si rods directly, while non-activated dyes need a linking agent such as EDC and Sulfo-NHS. By combining EDC and Sulfo-NHS, amine reactive Sulfo-NHS esters were created on any carboxyl-containing molecule. 19.2 mg EDC and 11.2 mg Sulfo/NHS was dissolved in 400 ul DI water and then mixed with 8 mg FITC, which had been dissolved in 100 μl DMSO. The mixture was magnetically stirred for 30 minutes and dropped onto APTES-treated Si nanorods and Si film (250 μl for each chip). The chips were left under room temperature for one hour and then transferred at 4° C. overnight.

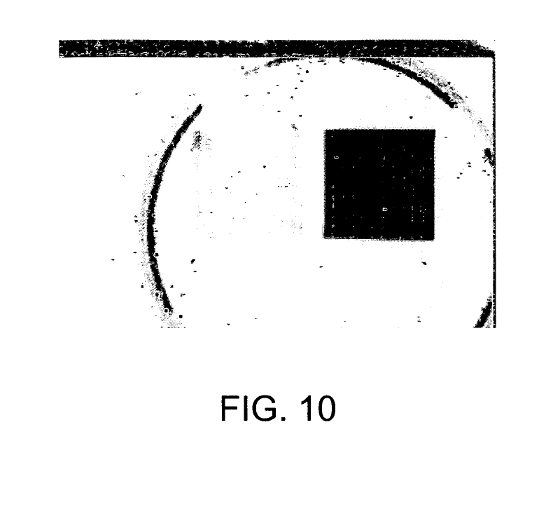

The chips were rinsed with DI water and dried under N₂. Images were taken from Typhon Scanner (488 nm excitation) (FIG. 10).

Integration of the intensity of FITC/Si nanorods (film) from the Typhoon scanner demonstrated an 8-fold enhancement with the nanorods.

| | intensity | enhancement |
|---|---|---|
| FITC/Si nanorods | 6422.365 | 8 |
| FITC/Si film | 813.768 | |

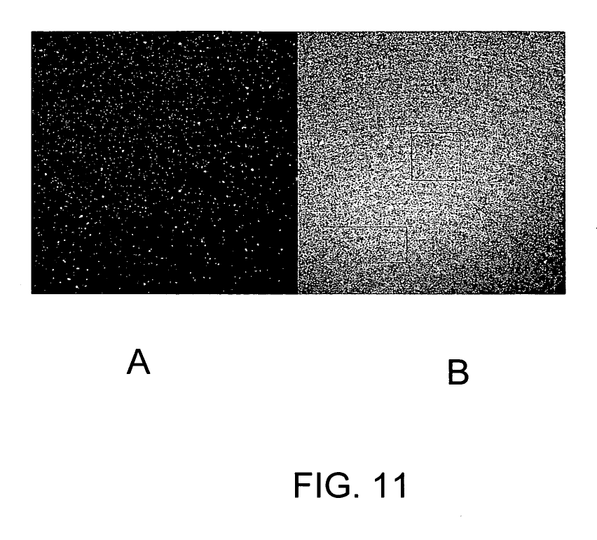

The 8-fold enhancement was in accord with the surface area ratio, which was also verified by the confocal microscope analysis. Confocal microscope images were taken under 488 nm excitation (FIG. 11).

Five images were taken for each chip. For each image, five regions were chosen for performing the integration of mean gray. The integration results are shown in the table below. According to the table, the enhancement is 8 fold.

| Region | Region | Region | Region | Region | |
|---|---|---|---|---|---|
| 1 | 2 | 3 | 4 | 5 | Average |

FITC/Si film

| | | | | | | |
|---|---|---|---|---|---|---|
| Image 1 | 21.83 | 18.48 | 24.85 | 15.76 | 11.05 | 18.394 |
| Image 2 | 20.33 | 23.96 | 26.44 | 34.37 | 23.93 | 25.806 |
| Image 3 | 16.70 | 20.96 | 20.21 | 19.78 | 15.70 | 18.67 |
| Image 4 | 13.79 | 19.43 | 21.72 | 24.76 | 19.50 | 19.84 |
| Image 5 | 14.12 | 12.88 | 17.76 | 20.60 | 17.91 | 16.654 |

| | |
|---|---|
| Total average | 19.87 |

FITC/Si nanorods

| | | | | | | |
|---|---|---|---|---|---|---|
| Image 1 | 71.64 | 101.39 | 110.31 | 185.55 | 197.72 | 133.322 |
| Image 2 | 129.83 | 166.47 | 199.93 | 236.42 | 199.12 | 186.354 |
| Image 3 | 99.30 | 130.55 | 163.21 | 212.61 | 163.22 | 153.778 |
| Image 4 | 102.27 | 135.02 | 172.64 | 212.42 | 157.41 | 155.952 |
| Image 5 | 145.71 | 168.29 | 211.02 | 193.04 | 118.19 | 167.25 |

| | |
|---|---|
| Total average | 159.33 |

Dye Conjugated Si Nanorods Solution

FITC conjugated Si nanorods were sonicated from the substrate into DI water. A 5 ul suspension was sandwiched between two 0.1 mm glass slides and observed by 60× fluorescence microscope (not shown). The Si nanorods were visible and exhibited Brownian Motion.

Dye-Conjugated Au/Si Nanorods Solution

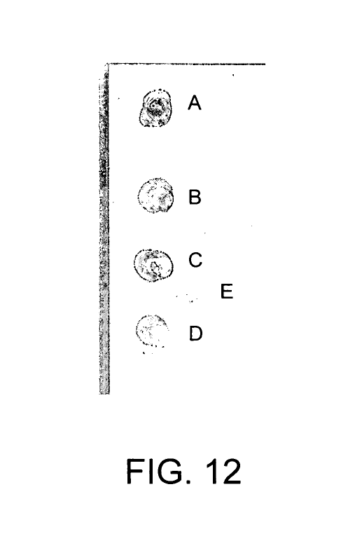

The gold-coated (Au/Si) nanorods conjugated with FITC were sonicated from the substrate into DI water and then transferred to the fluorescence microscope. Bright rods could be observed with low density. Later the nanorod suspensions were dropped on a glass slide and scanned under the Typhoon scanner (FIG. 12).

| | 1 | 2 | 3 | 4 | 5 |
|---|---|---|---|---|---|
| samples | Si | Au(15 nm)/ | Ag/Si | Au(7.5 nm)/ | DI |
| | nanorods/ | Si | nanorods/ | Si | water |
| | FITC | nanorods/ | FITC | nanorods/ | |
| | | FITC | | FITC | |
| intensity | 3963.2 | 2612.6 | 3403.9 | 2669.1 | 232.2 |

The signal from the Au/Si nanorods/FITC conjugation was slightly weaker than that from the Si nanorods/FITC, likely because part of the Si nanorods were covered by the gold layer. The results also included a sample of silver coated Si nanorods. This sample also gave a strong signal, probably due to metal enhanced fluorescence effects.

### Example 2

### Bio-Detection by Dye-Conjugated Au/Si Nanorods

Antibody Conjugation

Au/Si nanorods were fabricated on a chip as described in Example 1. A 5 mg/ml solution of dithiobis-succinimidyl propionate (DSP) (Pierce Chemical, Rockford, Ill.) in 100% DMSO (Sigma, St. Louis, Mo.) was spread over the gold coated Si nanorods surface and incubated for 30 minutes at room temperature. The chip was then rinsed with DMSO, followed by distilled water. The chip was immediately placed in a 1 mg/ml solution of RSV antibody in PBS, pH=7.4. After 2 hours incubation at room temperature, the chip was transferred to 4° C. overnight. The chip was then rinsed with PBS, followed by distilled water, and dried under a stream of nitrogen gas. To obtain the nanorods solution, antibody conjugated Au/Si nanorods/dye were sonicated from the substrate into PBS pH=7.4. During the sonification process, ice was added into the bath to maintain a low temperature.

RSV Detection

RSV infected cells and healthy cells were incubated in the wells. Equal amounts of the antibody conjugated Au/Si nanorods/dye dilutions were added into the wells and incubated for 2 hours. The wells were then washed with Tween/PBS 3 times and scanned by the Typhoon scanner.

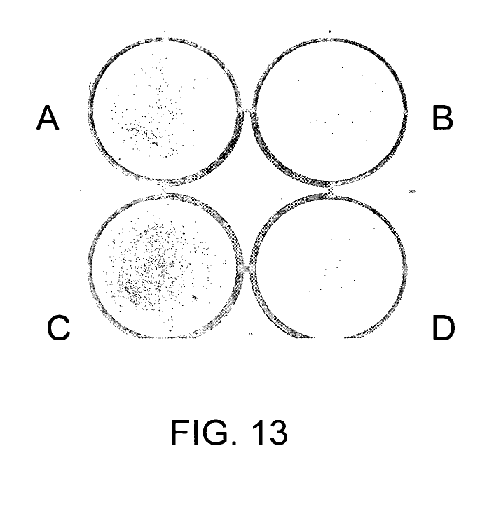

The results clearly show the specific staining of RSV infected cells and not the uninfected cells. The expected signal was present and indicated the successful detection of RSV infection by the antibody conjugated Au/dye/Si nanorods (FIG. 13).

We claim:

1. A composition for detecting a biomolecule of interest in a sample comprising:
a plurality of nanorods, wherein the nanorods include a binding agent having an affinity for the biomolecule of interest coupled to the surface of the nanorod and a reporter molecule coupled to the surface of the nanorod, wherein the reporter molecule is capable of providing a signal.

2. The composition of claim 1, wherein the biomolecule of interest is selected at least one of the following: a polypeptide, a protein, a glycoprotein, a nucleic acid, a carbohydrate, a lipid, a vitamin, a virus, a virus infected cell, and combinations thereof.

3. The composition of claim 1, wherein the biomolecule comprises an RNA or DNA virus.

4. The composition of claim 1, wherein the composition is selected from the following: a solution, a suspension, a gel, a sol gel, a colloid, and a combination thereof.

5. The composition of claim 1, wherein the sample is selected from one of the following: blood, saliva, tears, phlegm, sweat, urine, plasma, lymph, spinal fluid, cells, microorganisms, a combination thereof, and aqueous dilutions thereof.

6. The composition of claim 1, wherein the binding agent is selected from one of the following: a polynucleotide, a polypeptide, a protein, a glycoprotein, a lipid, a carbohydrate, a fatty acid, a fatty ester, a macromolecular polypeptide complex, and a combination thereof.

7. The composition of claim 6, wherein the polypeptide is an antibody or fragment thereof.

8. The composition of claim 3, wherein the virus is selected from the family of respiratory viruses including Orthomyxoviridae, Paramyxoviridae, adenoviruses, HIV, and a combination thereof.

9. The composition of claim 3, wherein the virus is respiratory syncytial virus (RSV).

10. The composition of claim 1, wherein the reporter molecule is a fluorescent dye molecule.

11. The composition of claim 10, wherein the fluorescent dye comprises an Alexa dye.

12. The composition of claim 1, wherein the nanorods comprise a material selected from at least one of the following materials: a metal, a metal oxide, a metal nitride, a metal oxynitride, a polymer, a multicomponent material, and combinations thereof.

13. The composition of claim 1, wherein the nanorods comprise silicon.

14. The composition of claim 1, wherein the nanorods comprise a metal layer coated on a portion of the surface of the nanorods.

15. The composition of claim 14, wherein the nanorod comprises at least one fluorescent dye molecule bound to the surface of the nanorod and a binding agent specific for the biomolecule of interest bound to the portion of the surface of the nanorod coated with the metal layer.

16. The composition of claim 14, wherein the metal layer comprises a ceramic material with electrical conductivity similar to that of metals.

17. The composition of claim 14, wherein the metal layer is selected from silver and gold.

18. The composition of claim 1, wherein the nanorods have a height of about 100 to 1000 nanometers, and a diameter of about 50 to 100 nanometers.

19. The composition of claim 14, wherein the metal layer is from about 20 to 100 nm thick.

20. A composition for detecting an analyte of interest in a sample comprising:
labeled nanorods, wherein the nanorods include a binding agent specific for the analyte of interest coupled to the surface of the nanorod and wherein the labeled nanorods are capable of providing a signal.

21. The composition of claim 20, wherein the analyte is selected from: biomolecules; toxins; drugs; viruses; cells containing toxings, drugs, or viruses; explosives; nuclear wastes; contaminants; biohazards; and combinations thereof.

22. A system for detecting a biomolecule of interest in a sample comprising:
a composition comprising labeled nanorods, wherein the nanorods include a binding agent having an affinity for the biomolecule of interest coupled to the surface of the nanorod and wherein the labeled nanorods are capable of providing a signal; and
a device for detecting the signal produced by the labeled nanorods to determine the presence or absence of the biomolecule of interest.

23. The system of claim 22, wherein the signal is a fluorescence signal and the detecting device comprises a device capable of detecting fluorescence selected from: a fluorescence microscope, a confocal fluorescence microsocpe, fluorescence spectroscopy, and a fluorescence scanner.

24. A method of making a composition of nanorods for detecting a biomolecule of interest in a sample comprising:
providing a substrate;
depositing an array of nanorods on the substrate by galancing angle vapor deposition;
labeling the nanorods by immobilizing a reporter molecule onto at least a portion of the surface of each nanorod;
immobilizing a binding agent having an affinity for the biomolecule of interest onto a portion of the surface of each nanorod; and
removing the nanorods from the substrate to form a composition of nanorods.

25. The method of claim 24, wherein further comprising rotating the substrate in a polar direction relative to a vapor arrival line of a vapor flux of a material to achieve a desired incident angle φ defined by the vapor arrival line and the surface normal of the planar substrate.

26. The method of claim 25, wherein the substrate is planar, and wherein the incident angle φ is greater than about 75°.

27. The method of claim 26, wherein φ is between about 75° and about 89°.

28. The method of claim 26, wherein φ is about 86°.

29. The method of claim 24, the nanorods comprise a material selected from:
a metal, a metal oxide, a metal nitride, a metal oxynitride, a polymer, a multicomponent material, and combinations thereof.

30. The method of claim 29, wherein the nanorods comprise silicon.

31. The method of claim 24, further comprising depositing a metal layer onto at least a portion of the surface of each nanorod.

32. The method of claim 31, wherein the metal layer comprises a metal selected from: silver and gold.

33. The method of claim 31, wherein the metal layer is on an end of the nanorods that is not attached to the substrate.

34. The method of claim 31, wherein the binding agent is immobilized to the metal coated portion of the nanorod.

35. The method of claim 31, wherein the metal layer is applied to the nanorods via a process selected from one of: sputtering growth, chemical vapor deposition, and thermal evaporation.

36. The method of claim 24, wherein the reporter molecule is a fluorescent dye.

37. The method of claim 34, wherein the dye is an Alexa dye.

38. The method of claim 24, wherein the binding agent is selected from one of the following: a polynucleotide, a polypeptide, a protein, a glycoprotein, a lipid, a carbohydrate, a fatty acid, a fatty ester, a macromolecular polypeptide complex, or a combination thereof

39. The method of claim 24, wherein the binding agent is an antibody.

40. The method of claim 24, wherein the biomolecule of interest is selected from one of the following: a polypeptide, a protein, a glycoprotein, a nucleic acid, a carbohydrate, a lipid, a vitamin, a virus, a virus infected cell, or a combination thereof.

41. The method of claim 40, wherein the virus is selected from the family of respiratory viruses including Orthomyxoviridae, Paramyxoviridae, adenoviruses, HIV, or a combination thereof.

42. The composition of claim 40, wherein the virus is respiratory syncytial virus (RSV).

43. The method of claim 24, wherein the nanorods are removed from the substrate via a process selected from one of: sonication, mechanical ablation, selective chemical desorption, and selective substrate corrosion.

44. The method of claim 24, wherein the composition of nanorods is selected from: a solution, a suspension, a colloid, a gel, a sol gel, and a combination thereof.

45. The method of claim 24, wherein one or more of the binding agent and the reporter molecule is disposed on the nanorods via a linking agent.

46. The method of claim 45 wherein the linking agent includes linking agents selected from: dithiobis(succinimidyl propionate) (DSP), a self-assembly monolayer (DSP), and 3-Aminopropyl)triethoxysilane (APTES).

47. A method for detecting a biomolecule of interest in a sample comprising:
contacting the sample with a composition comprising a plurality of labeled nanorods, wherein the nanorods include a binding agent having an affinity for the biomolecule of interest, wherein the labeled nanorods are capable of providing a detectable signal, and wherein, in the presence of the biomolecule of interest, the labeled nanorods bind the biomolecule of interest; and
detecting the signal produced by the labeled nanorods to determine the presence or absence of the biomolecule of interest.

48. The method of claim 47, further comprising removing unbound nanorods from the sample.

49. The method of claim 47, wherein the labeled nanorods comprise a reporter molecule coupled to the surface of the nanorod, and wherein the reporter molecule provides the detectable signal.

50. The method of claim 49, wherein the reporter molecule is a fluorescent dye.

51. The method of claim 47, wherein the biomolecule of interest comprises a cell infected with a virus.

52. The method of claim 50, wherein the virus is respiratory syncytial virus (RSV).

53. The method of claim 47, wherein the binding agent is an antibody.

54. A composition for detecting a biomolecule of interest in a sample comprising:
a plurality of nanostructures, wherein the nanostructures include a binding agent having an affinity for the biomolecule of interest coupled to the surface of the nanostructure and a reporter molecule coupled to the surface of the nanostructure, wherein the reporter molecule is capable of providing a signal.

55. The composition of claim 54, wherein the nanostructure is selected from: nanorods, nanospheres, nanowires, nanotubes, nanospirals, and combinations thereof.
